(19) United States
(12) Patent Application Publication
(10) Pub. No.: US 2018/0105851 A1
(43) Pub. Date: Apr. 19, 2018

(54) METHOD AND SYSTEM FOR PREPARING PULP FOR PAPER WITH GRASS STRAWS AS RAW MATERIAL

(71) Applicant: DALIAN SANYUANXIN ENZYMATIC SCIENCE & TECHNOLOGY CO., LTD., DALIAN (CN)

(72) Inventors: HUA SUN, DALIAN (CN); ZHONGYU LIU, DALIAN (CN)

(21) Appl. No.: 15/604,595

(22) Filed: May 24, 2017

(30) Foreign Application Priority Data
Oct. 14, 2016 (CN) 201610900493.3

(51) Int. Cl. C12P 19/02; D21H 11/12; D21C 5/00; D21C 7/00; C12P 19/14

(52) U.S. Cl. CPC C12P 19/02; D21H 11/12; D21C 5/005; C12P 2201/00; C12P 19/14; C12Y 302/01008; C12Y 302/01078; D21C 7/00

(57) ABSTRACT

The present invention provides a method and a system for preparing pulp for paper with grass straws as raw material. The method of the present invention includes the following steps: 1) adding water in raw material for pulping, performing a first enzymolysis with ligninolytic enzyme; 2) cooking the first enzymolysis product at 80-130° C.; 3) performing second enzymolysis with hemicellulase to the cooked product. The method of the present invention is able to improve the pulp yield, reduce discharge of pollutants from black liquor, lower alkali consumption and energy consumption, and facilitate extraction of lignin and C-5 sugars, and pulp has a high quality.

### CROSS-REFERENCE TO RELATED APPLICATIONS

This application claims priority to Chinese Patent Application No. 201610900493.3, filed on Oct. 14, 2016, entitled “METHOD AND SYSTEM FOR PREPARING PULP FOR PAPER WITH GRASS STRAWS AS RAW MATERIAL”, which is incorporated herein by reference in its entirety.

### TECHNICAL FIELD

The present invention relates to a pulping process, and specifically relates to a method and a system for preparing pulp for paper and paper products with grass straws as raw material.

### BACKGROUND

Paper is an indispensable and important part of everyday life for humans. Paper pulp is mainly produced from the logging of trees or the harvest of crop straws. As the growth rate of forests cannot meet increasing global demand for paper products, and as an effective way to reduce deforestation and protect the environment, utilization of reproducible crop straws as a raw material for paper pulp is a readily available alternative

Currently, the dominant method for employing crop straws to produce paper pulp is an alkali cooking method, in which the production process is carried out as the following steps: “cooking-pulp grinding-pulp washing-screening”; specifically, the method typically uses alkali with a mass content of 14-18% to cook crop straws for 45 min-1.5 h in 160-180° C. high-pressure steam, so as to obtain the pulp. In the above method, hemicellulose and lignin are exfoliated from the outer portion of straws fibers under the action of high-concentration alkali, so as to obtain the pulp under the high temperature and high pressure, however, pulp yield is compromised due to damage caused to part of the cellulose from the strong alkali environment as the alkali plays its role; at the same time, due to the action of the strong alkali, lignin, hemicellulose and the like are decomposed into small molecule substances, thus forming a black liquor, which tends to color the cellulose black, thereby reducing brightness of the pulp; and the requirement for the brightness entails adding steps such as delignification, thereby improving the cost of pulping. Additionally, cooking at high temperature with a strong alkali will make lignin and C-5 sugars derived from hemicellulose dissolve into inseparable black liquor, causing environmental pollution, and meanwhile making it impossible to extract the lignin and C-5 sugars.

### SUMMARY

The present invention provides a method and system for preparing pulp for paper with grass straws (wheat, rice, oats, barley, switch grass, etc.) as raw material, the method may adopt a “enzymolysis-cooking-enzymolysis-dewatering-enzymolysis” process and can be conducted by the system, the process is able to reduce alkali consumption and energy consumption, improve pulp yield, as well as facilitate extraction of lignin and C-5 sugars.

The present invention provides a method for preparing pulp for paper (i.e., the paper pulp) with grass straws as raw material, including the following steps:

1) adding water or recycled process water into a raw material for pulping to form a mixture, and performing a first enzymolysis to the mixture with a ligninolytic enzyme to obtain a first enzymolysis product;

2) cooking the first enzymolysis product at a temperature of 80-130° C. to obtain a cooked product;

3) performing a second enzymolysis to the cooked product with hemicellulase to obtain a second enzymolysis product.

The pulping method of the present invention imposes no strict limits on the raw material for pulping, which may be conventional raw material for pulping in this field, especially crop straws, such as rice straws, wheat straws and other grass straws.

Further, the first enzymolysis may be performed after a pretreatment of the raw material for pulping; the pretreatment may include impurity removal, which may be removing impurities like dust, leaves and others with low content of cellulose in the raw material for pulping; the pretreatment may also include a cutting process, which may be preparing the raw material for pulping into segments with specific size, for example, the raw material for pulping may be cut into segments with 80% of which have a length less than 25 mm, thus contributing to improvement in subsequent processing effects.

In the present invention, the first enzymolysis is used for breaking fiber surface structure of the raw material for pulping, so as to reduce the temperature of the subsequent cooking, and to reduce the amount of alkali used in the cooking and the discharge of black liquor; there is no strict limitation of specific type of the ligninolytic enzyme, for example, the ligninolytic enzyme may be one or more of lignin peroxidase, manganese peroxidase, and laccase, etc.; in an embodiment of the present invention, the ligninolytic enzyme is laccase, especially laccase produced by White Rot Fungi.

Further, in the above step 1), a mass ratio of the raw material for pulping (dry material) to water or recycled process water is 1: (5-10), for example, 1:6; wherein the water content of the raw material for pulping (dry material) is 9 wt %-16 wt %; in addition, there are no strict limits on the enzyme activity and amount of the ligninolytic enzyme, which may be properly set according to practical needs, and the enzyme activity of the ligninolytic enzyme may be, for example, 8,000-12,000 U/g, the amount of the ligninolytic enzyme may be 0.05-0.2 kg per 100 kg of raw material for pulping and the water or recycled process water. Additionally, there are no strict limits on conditions for the first enzymolysis, which may be conventional conditions in this field, for example, the temperature of the first enzymolysis may be controlled at 40-80° C., pH value may be 5-8, and enzymolysis time may be 0.5-4 h.

After the above first enzymolysis, the amount of organic matter in the first enzymolysis product accounts for about 8-12 wt % of absolute dry weight of the raw material, the dissolution rate of lignin is 57.8%-73.1%, and the dissolution rate of C-5 sugars is 40.6%-48.8%; thus the temperature of the subsequent cooking as well as alkali consumption and energy consumption can be significantly reduced, the pulp yield can be improved. The dissolution rate of a substance in the present invention refers to the proportion (by percentage) of the substance released from the raw material for pulping; for example, the dissolution rate of lignin being 57.8%-73.1% means 57.8%-73.1% of lignin in the raw material for pulping is released. Additionally, research has shown that: the temperature required by the subsequent cooking and alkali consumption would have been significantly increased, if the above first enzymolysis has not been performed.

In the present invention, cooking is used for separating hemicellulose and lignin in the raw material for pulping, thereby forming a pulp. Specifically, an alkali liquor may be adopted for performing the cooking, and there are no strict limits on the mass content of the alkali liquor. Additionally, the amount of the alkali liquor added may be controlled such that the pH value of the solution to be cooked is 8-12, specifically 8-10, and more specifically 8-9; and the time for the cooking may is 0.5-1.5 h.

After the above cooking, the amount of hemicellulose in the cooked product is 42 wt %-46 wt %, the dissolution rate of carbohydrate reaches 22% or so. In the present invention, the small amount of alkali used in the cooking, low processing temperature and short processing time are helpful to avoid damages to the fiber, which in turn improve the pulp yield, and further reduce alkali consumption and the discharge of black liquor, avoid environmental pollution, save thermal energy, and are particularly conducive to the extraction of lignin and C-5 sugars, thereby avoiding waste of resources.

In the present invention, the second enzymolysis is used for separating lignin among the celluloses in cell walls as well as other non-fiber components, which are difficult to be separated in the raw material for pulping; there are no strict limits for the hemicellulase, which may be a conventional hemicellulase in this field, for example, the hemicellulase can be one or more of xylanase, galactosidase, and mannase, etc. Additionally, there are no strict limits for conditions of the second enzymolysis, which may be properly set in accordance with the type of the used hemicellulase.

Specifically, in steps 3), the hemicellulase may include xylanase and mannase, and the mass ratio of the xylanase and the mannase in the hemicellulase may be (6-8):(2-4), for example, 7:3.

Further, in steps 3), enzyme activities of both the xylanase and the mannase may be 8,000-10,000 U/g, the amounts of xylanase and mannase are both 0.05-0.2 kg per 100 kg of the cooked product; and the temperature of the second enzymolysis may be controlled at 40-70° C., pH value may be 8-12, and enzymolysis time may be 0.5-4 h.

In the above second enzymolysis products, more than 70% of the dissolved lignin and lignin-carbohydrate composite (LCC) have a relative molecular weight of 2,000-10,000, thus facilitating subsequent extraction and utilization; additionally, the dissolution rate of C-5 sugars in the second enzymolysis is 48% or so; the dissolution rate of carbohydrate is 48% or so; the alkali consumption amount is about 73 wt %. Additionally, research has shown that: if the above second enzymolysis is not performed, lignin among the celluloses in cell walls (which is difficult to be separated) will not able to be effectively removed from the raw material for pulping, and the pulp produced thereby will have a poor quality.

The method of the present invention may also include step 4): performing dewatering to the second enzymolysis product, and performing a third enzymolysis to the dewatered product with hemicellulase. There are no strict limits imposed by the present invention to the hemicellulase in step 4), which may be one or more conventional hemicellulases in this field. Further, the hemicellulase in step 4) and the hemicellulase in steps 3) may be the same or different.

Specifically, in step 4), the hemicellulase includes xylanase and mannase, and the mass ratio of the xylanase to the mannase in the hemicellulase is (4-6):(6-4).

Further, enzyme activities of the xylanase and the mannase are both 8,000-10,000 U/g, and the amounts of xylanase and mannase are both 0.05-0.2 kg per 100 kg of the dewatered product; and the temperature of the third enzymolysis is controlled at 40-70° C., pH value is 8-12, and enzymolysis time is 1-4 h.

Further, in step 4), performing a third enzymolysis to the dewatered product with a mixture of hemicellulase and ligninolytic enzyme, wherein enzyme activity of the ligninolytic enzyme is 8,000-12,000 U/g, and the amount of the ligninolytic enzyme is 0.01-0.1 kg per 100 kg of the dewatered product.

Additionally, in step 4), extraction of lignin and/or C-5 sugars may also be performed to the liquid removed during the dewatering process, so as to avoid waste of resources; further, the liquid after extraction of lignin and/or C-5 sugars may be recycled via evaporation-condensation, thus reducing water consumption, cutting or eliminating discharge of waste water, and improving water efficiency.

In the above third enzymolysis, the dissolution rate of lignin is higher than 94%, the dissolution rate of C-5 sugars is 52%-58%, and in the third enzymolysis product, the Benzyl alcohol extract is less than 5 wt %, and the Kappa number is 13-18.

Further, as practically needed, the above third enzymolysis product may be further subjected to pulp washing, pulp grinding, screening and drying; particularly, when high brightness is required, bleaching may be performed as needed before the drying.

The present invention also provides a system (i.e., a pulping system) for implementing any of the above methods, the system including a first enzymolysis device, a cooking device and a second enzymolysis device which are sequentially connected; wherein the first enzymolysis device is used for performing the first enzymolysis, the cooking device is used for performing the cooking, and the second enzymolysis device is used for performing the second enzymolysis.

Further, the system may also include:

a dewatering device, which is connected with the second enzymolysis device and is used for performing the dewatering;

a third enzymolysis device, which is connected with the dewatering device and is used for performing the third enzymolysis.

Further, the system may also include:

a preparatory device which is connected with the first enzymolysis device and is used for performing the pretreatment to the raw material for pulping.

In the system of the present invention, all the devices may be conventional devices in this field.

Implementation of the present invention at least has the following advantages:

1. In the method of the present invention, performing the first enzymolysis to the raw material for pulping before the cooking breaks fiber surface structure of the raw material for pulping, which is advantageous for reducing the temperature of the subsequent cooking and the amount of alkali during the cooking, and the alkali consumption per ton of pulp can be reduced by 50-70 wt %.

2. During the cooking of the present invention, the small alkali consumption, low processing temperature and short processing time are conducive to avoiding damages to the fiber, which in turn improve pulp yield by 5-10%, and further reduce the discharge of black liquor and pollutants, avoid environmental pollution, save thermal energy, and are particularly conducive to the extraction of lignin and C-5 sugars, thereby avoiding waste of resources.

3. In the present invention, performing the second and the third enzymolysis to the cooked product further reduces the content of lignin in the pulp, thus guaranteeing the brightness of the pulp liquid, so that the bleaching process may be omitted when producing unbleached pulp, which facilitates water saving, and the water consumption may be reduced by 60-80% by volume.

4. In the method of the present invention, crude pulp yield is higher than 50%; Kappa number of the pulp is 9-13; the dissolution rate of lignin is up to 95% or so; the dissolution rate of C-5 sugars reaches 50% or higher; the cost per ton of pulp is less than half of that in a conventional method, as a result, the present invention is provided with higher economic benefits.

5. The pulp prepared according to the method of the present invention has a brightness of near 60%, a folding endurance up to 38 times, a tearing index of 4 mN·m²/g or so, and a tensile index of 48 N·m/g, and also retains fine fibers in the raw material for pulping, overcomes the deficiency of poor water filterability of the conventional pulp (straw pulp). The pulp has good papermaking performance, and main indices of the fiber are on a par with those of hardwood pulp.

### BRIEF DESCRIPTION OF THE DRAWINGS

FIG. 1 is a structural diagram of a pulping system in an embodiment of the present invention.

### DETAILED DESCRIPTION

In order to make the purpose, technical solutions and advantages of the present invention clearer, the technical solutions of embodiments of the present invention will be clearly and completely described in conjunction with the embodiments of the present invention. Apparently, the described embodiments are part rather than all of the embodiments of the present invention. All the other embodiments obtained by one with ordinary skill in the art on the basis of the embodiments of the present invention without delivering creative efforts shall fall into the protection scope of the present invention.

Enzymes adopted in the following embodiments are purchased from Dalian San Yuan Xin Enzymatic & Biotech Co. Ltd., wherein: the laccase is a laccase of No. DLQ-02 and with enzyme activity of 8,000-12,000 U/g; the xylanase is a xylanase of No. SH-01 and with enzyme activity of 8,000-10,000 U/g; and the mannase is a mannase of No. SH-05 and with enzyme activity of 8,000-10,000 U/g.

### Embodiment 1

1. Pretreatment

Cutting rice straws (i.e., raw material for pulping, with water content of 10-14 wt %) that tied up as a circular or square bundle into segments, wherein 80% of the segments have a length of less than 25 mm; and removing impurities such as sand, dust, leaves and others with low content of cellulose in the segments of rice straws, so as to obtain pretreated rice straws.

2. First Enzymolysis

Mixing the pretreated rice straws with water at a mass ratio of 1:6 to form a mixture, adjusting the pH value of the mixture to 6 or so, heating to about 40° C., maintaining the temperature, adding the laccase with enzyme activity of 10,000 U/g into the mixture to perform a first enzymolysis, wherein the amount of the laccase is 0.1% (i.e., 0.1 kg laccase/100 kg the mixture of the pretreated rice straws and the water); and performing the first enzymolysis for about 2 h, so as to obtain a first enzymolysis product.

Detecting the amount of lignin in the solution of the first enzymolysis product with ultraviolet-visible spectrophotometry, the result of which shows that: after the first enzymolysis, the dissolution rate of lignin in the raw material is about 57.8%; and the dissolution rate of C-5 sugars is about 45.03%. Observation of the first enzymolysis product with an electron microscope suggests that, stratum corneum of the fiber cells in the raw material has already been broken, and serrated cells are present in the product, indicating that the first enzymolysis is able to break the fiber surface structures of the raw material for pulping.

3. Cooking

Adding sodium hydroxide solution into the first enzymolysis product, so as to adjust pH value of the first enzymolysis product to about 8-9, then heating to 110° C. or so, cooking the first enzymolysis product for about 1 h, a cooked product is obtained; through detection, the amount of lignin in the cooked product is 9 wt %, the dissolution rate of C-5 sugars is about 50%, and the fiber is softening and swelling. During observation of the cooked product with an electron microscope, it can be seen that a large number of miscellaneous cells (for example, epidermal cells, parenchyma cells and the like) are dissolved, suggesting that internal structure of the raw material is decomposed in the cooking process.

4. Second Enzymolysis

Adjusting pH value of the cooked product to 9 or so, cooling to about 50° C., maintaining the temperature, adding mixed enzyme of the xylanase and the mannose into the cooked product to perform a second enzymolysis, wherein the mass ratio of the xylanase to the mannase in the mixed enzyme is 7:3, enzyme activities of both the xylanase and the mannase are both 9,000 U/g or so, and the amount of the mixed enzyme is 0.2% (i.e., 0.2 kg mixed enzyme/100 kg cooked product); and performing the second enzymolysis for about 1 h, so as to obtain a second enzymolysis product; through detection, the amount of lignin in the second enzymolysis product is 6 wt %, the dissolution rate of carbohydrate reaches higher than 30%, and the dissolution rate of C-5 sugars is 50% or so. During observation of the second enzymolysis product with an electron microscope, plant miscellaneous cells and fine fibers are spotted across the whole vision, and condensed lignin is present, suggesting that in the raw material for pulping, lignin among the celluloses in cell walls (which is difficult to be separated) is separated during the second enzymolysis.

5. Third Enzymolysis

Performing dewatering to the second enzymolysis product to obtain dewatered material (i.e. the dewatered product), the removed water may further be subjected to extraction of lignin, C-5 sugars and the like, and the water after extraction of lignin and C-5 sugars may be recycled via evaporation-condensation.

Mixing the dewatered material with water at a mass ratio of 1:6 to form a mixture, adjusting pH value of the mixture to 8 or so, heating to about 45° C., maintaining the temperature, adding mixed enzymes of the xylanase and the mannase as well as the laccase to the mixture to perform a third enzymolysis, wherein the mass ratio of the xylanase to the mannase in the mixed enzymes is 1:1, enzyme activities of the xylanase and the mannase are both 9,000 U/g or so, the amounts of the xylanase and mannase are both 0.1 kg per 100 kg of the dewatered material, the amount of laccase is 0.05 kg per 100 kg of the dewatered material, performing the third enzymolysis for about 2 h, thus obtaining a third enzymolysis product (i.e., a pulp), which may subsequently be delivered for pulp washing, pulp grinding, screening, pulp sheet drying, packaging, transportation and the like.

After the third enzymolysis, the dissolution rate of lignin is 94.73%, and the dissolution rate of C-5 sugars is 52.92% through detection.

Additionally, detecting brightness of the pulp with a China GB/T 8940.2-2002 method; detecting the folding endurance, the tearing strength and the tensile strength of the pulp with a China GB/T 24323-2009 method; detecting ash content of the pulp with a China GB/T 742-2008 method; and detecting fiber length of the pulp with a China GB/T 29779-2013 method, the results of which are shown in table 1.

### Embodiment 2

1. Pretreatment

Cutting wheat straws (with water content of 12 wt %) into segments, so that more than 80% of the segments have a length of less than 25 mm; and removing impurities such as sand, dust, leaves and others with low content of cellulose from the segments of wheat straws, thus obtaining the pretreated wheat straws.

2. First Enzymolysis

Mixing the pretreated wheat straws with water at a mass ratio of 1:6 to form a mixture, adjusting pH value of the mixture to 5 or so, heating to 50° C. or so, maintaining the temperature, adding a laccase with enzyme activity of 10,000 U/g into the mixture to perform a first enzymolysis, wherein the amount of the laccase is 0.05 kg per 100 kg the mixture of the pretreated wheat straws and the water; and performing the first enzymolysis for 2 h or so, thus obtaining a first enzymolysis product; after the first enzymolysis, the dissolution rate of lignin is 73%, and the dissolution rate of C-5 sugars is 40% through detection.

3. Cooking

Adding sodium hydroxide solution into the first enzymolysis product, so that pH value of the first enzymolysis product reaches 12 or so, then heating to about 120° C., maintain the temperature, cooking the first enzymolysis product for 1.5 h or so; and after the cooking, a cooked product is obtained, the dissolution rate of lignin is 90%, and the dissolution rate of C-5 sugars is 49.1% through detection.

4. Second Enzymolysis

Adjusting pH value of the cooked product to 8 or so, heating temperature to 40° C. or so, maintaining the temperature, adding mixed enzymes of a xylanase and a mannase into the cooked product to perform a second enzymolysis, wherein the mass ratio of the xylanase to the mannase in the mixed enzymes is 6:4, enzyme activities of the xylanase and the mannase are both 9,000 U/g or so, the amount of the mixed enzymes is 0.2 kg per 100 kg of the cooked product; performing the second enzymolysis for 2 h or so, thus obtaining a second enzymolysis product; and after the second enzymolysis, the dissolution rate of lignin is 92.9%, and the dissolution rate of C-5 sugars is 52% through detection. During observation of the second enzymolysis product with an electron microscope, mostly observed are straw miscellaneous cells and fine fibers.

5. Third Enzymolysis

Performing dewatering to the second enzymolysis product to obtain dewatered material (i.e. the dewatered product), the removed water may further be subjected to extraction of lignin, C-5 sugars and the like, and the water after extraction of lignin and C-5 sugars may be recycled via evaporation-condensation.

Mixing the dewatered material with water at a mass ratio of 1:5 to form a mixture, adjusting pH value of the mixture to 9 or so, heating to about 40° C., maintaining the temperature, adding mixed enzymes of the xylanase and the mannase as well as the laccase to the mixture to perform a third enzymolysis, wherein the mass ratio of the xylanase to the mannase in the mixed enzymes is 6:4, enzyme activities of the xylanase and the mannase are both 9,000 U/g or so, the amounts of the xylanase and the mannase are both 0.05 kg per 100 kg of the dewatered material, and the amount of the laccase is 0.1 kg per 100 kg of the dewatered material; performing the third enzymolysis for 1 h or so, thus obtaining a third enzymolysis product (i.e., a pulp), which may subsequently be delivered for pulp washing, pulp grinding, screening, pulp sheet drying, packaging, transportation and the like. And the processes and quality test results of the pulp are shown in 1.

### Embodiment 3

1. Pretreatment

Cutting rice straws (with water content of about 12 wt %) into segments, so that more than 80% of the segments have a length of less than 25 mm; and removing impurities such as sand, dust, leaves and others with low cellulose content in the segments of rice straws, so as to obtain pretreated rice straws.

2. First Enzymolysis

Mixing the pretreated wheat straws with water at a mass ratio of 1:8 to form a mixture, adjusting pH value of the mixture to 7 or so, heating to 60° C. or so, maintaining the temperature, adding a laccase with enzyme activity of 10,000 U/g into the mixture to perform a first enzymolysis, wherein the amount of the laccase is 0.2 kg per 100 kg mixture of the pretreated rice straws and water; and performing the first enzymolysis for 1 h or so, thus obtaining a first enzymolysis product; after the first enzymolysis, the dissolution rate of lignin is 58%, and the dissolution rate of C-5 sugars is 40% through detection.

3. Cooking

Adding sodium hydroxide solution into the first enzymolysis product, so that pH value of the first enzymolysis product reaches 9 or so, then heating to about 130° C., maintain the temperature, cooking the first enzymolysis product for 0.5 h or so; and after the cooking, a cooked product is obtained, the dissolution rate of lignin is 77%, and the dissolution rate of C-5 sugars is 49.04% through detection.

4. Second Enzymolysis

Adjusting pH value of the cooked product to 10 or so, heating to 70° C. or so, maintaining the temperature, adding mixed enzymes of a xylanase and a mannase into the cooked product to perform a second enzymolysis, wherein the mass ratio of the xylanase to the mannase in the mixed enzymes is 8:2, enzyme activities of the xylanase and the mannase are both 9,000 U/g or so, the amount of the mixed enzymes is 0.2 kg per 100 kg of the cooked product; performing the second enzymolysis for 1 h or so, thus obtaining a second enzymolysis product; and after the second enzymolysis, the dissolution rate of lignin is 93%, and the dissolution rate of C-5 sugars is 51% through detection.

5. Third Enzymolysis

Performing dewatering to the second enzymolysis product to obtain dewatered material (i.e. the dewatered product), the removed water may further be subjected to extraction of lignin, C-5 sugars and the like, and the water after extraction of lignin and C-5 sugars may be recycled via evaporation-condensation.

Mixing the dewatered material with water at a mass ratio of 1:8 to form a mixture, adjusting pH value of the mixture to 9 or so, heating to about 60° C., maintaining the temperature, adding mixed enzymes of the xylanase and the mannase as well as the laccase to the mixture to perform a third enzymolysis, wherein the mass ratio of the xylanase to the mannase in the mixed enzymes is 6:4, enzyme activities of the xylanase and the mannase are both 9,000 U/g or so, the amounts of the xylanase and the mannase are both 0.1 kg per 100 kg of the dewatered material, and the amount of the laccase is 0.05 kg per 100 kg of the dewatered material; performing the third enzymolysis for 3 h or so, thus obtaining a third enzymolysis product (i.e., a pulp), which may subsequently be delivered for pulp washing, pulp grinding, pulp sheet drying, packaging, transportation, and the like. And the processes and quality test results of the pulp are shown in 1.

### Description of Processes in the Embodiments

The pulping system of the present invention includes a first enzymolysis device, a cooking device and a second enzymolysis device which are sequentially connected; wherein the first enzymolysis device is used for performing the first enzymolysis, the cooking device is used for performing the cooking, and second enzymolysis device is used for performing the second enzymolysis.

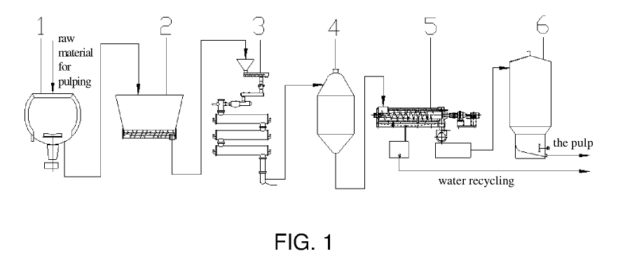

Further, as shown in FIG. 1, the pulping system used for any of the methods in embodiments 1-3 includes a pretreatment device 1, a first enzymolysis device 2, a cooking device 3, a second enzymolysis device 4, a dewatering device 5, and a third enzymolysis device 6 which are sequentially connected.

Specifically, placing the cut raw material for pulping in the pretreatment device 1 for the pretreatment, wherein the pretreatment device 1 is used for removing impurities such as sand, dust, leaves and others with low content of cellulose in the raw material for pulping, and the pretreatment device 1 may be a dry processing device or a wet processing device; after the pretreatment, feeding the raw material for pulping into the first enzymolysis device 2, where the ligninolytic enzyme breaks fiber surface structures of the raw material for pulping, so as to reduce the temperature during the subsequent cooking, and to reduce the amount of alkali used in the cooking and the discharge of black liquor; after the first enzymolysis, feeding the first enzymolysis product into the cooking device 3, where hemicellulose and lignin in the raw material for pulping are separated, and the cooking device 3 may be a spherical digester and a material feeding system thereof, and may also be a continuous cooking digester device and a material feeding system thereof, or a vertical digester and a material feeding system thereof; feeding the cooked product into the second enzymolysis device 4 where lignin and other non-fiber components are separated; feeding the second enzymolysis product into the dewatering device 5 for dewatering, wherein the removed liquid may undergo extraction of lignin and C-5 sugars, the water after extraction of lignin and C-5 sugars may be recycled via evaporation-condensation; and feeding the dewatered material into the third enzymolysis device 6 for the third enzymolysis, so as to further remove lignin and other non-fiber components, and the third enzymolysis product may be delivered to subsequent process such as pulp washing, pulp grinding, pulp sheet drying, packaging and transportation, and the like.

The above system may be employed to implement the pulping process of “enzymolysis-cooking-enzymolysis-dewatering-enzymolysis”, which is capable of producing virgin pulp of grass straws with low alkali consumption and high brightness.

### Comparative Example 1

Mixing the pretreated rice straws in embodiment 1 with water at a mass ratio of 1:6 to form a mixture, adding sodium hydroxide solution with a mass content of 20%, so that pH value of the mixture reaches 14 or so, cooking the mixture for about 1 h at about 160° C. in high-pressure steam, thus obtaining a pulp, the amount of lignin therein is 4.83 wt %, the dissolution rate of C-5 sugars is 54.98%, and the specific quality test results thereof are shown in table 1.

Additionally, in comparison with the method of comparative example 1, for one ton of pulp, the alkali consumption may be reduced by 50-70 wt % and water consumption may be reduced by 60-80% by volume, the pulp yield may be improved by 11%, and the cost may be lowered by more than 50% in the embodiments 1-3 methods of the present invention.

TABLE 1
Processes and quality test results of the pulp

| | | Pulp in | Pulp in | Pulp in |
|---|---|---|---|---|
| | | embodi- | embodi- | embodi- |
| Items detected | | ment 1 | ment 2 | ment 3 |
| Yield of crude pulp, % | | 54.00 | 52.68 | 50.48 |
| Kappa number | | 18 | 15 | 13 |
| Dissolution rate of lignin, % | | 94.73 | 95.17 | 95.40 |
| Dissolution rate of C-5 sugars, % | | 52.92 | 54.98 | 53.74 |
| Brightness, % | | 45.60 | 56.10 | 47.20 |
| Folding endurance, times | | 29 | 38 | 32 |
| Tearing index, mN · m²/g | | 3.5 | 4.1 | 3.9 |
| Tensile index, N · m/g | | 38.6 | 48.5 | 41.0 |
| Ash content (900° C.), % | | 5.2 | 4.7 | 4.9 |
| Fiber length, mm | Arithmetic Average | 0.54 | 1.05 | 0.66 |
| | Fiber length | | | |
| | Weighted Average | 0.82 | 1.53 | 0.91 |
| | length | | | |
| | Weight Weighted | 1.27 | 1.85 | 1.33 |
| | Average Fiber | | | |
| | length | | | |

It can be concluded from table 1 that:

The pulps prepared in the embodiments of the present invention have high brightness, good folding endurance, and a high tearing index and tensile index; extracting fibers of the straws in a relatively mild environment avoids degradation of fibers caused by strong alkalis at a high temperature and surface damage of the fibers, thereby protecting most of the fine fibers and avoiding over degradation of hemicellulose, thus improving pulp yield during the pulping, overcoming the deficiency of poor water filterability of conventional grass pulp (such as the pulp in comparative example 1). In such a way, the pulp obtained by the pulping method of the present invention can have good papermaking performance, the main indices of the fiber can be equivalent to those of hardwood pulp, and the pulp has an excellent quality.

Finally, it should be noted that: the above embodiments are merely intended to illustrate rather than limit the technical solutions of the present invention; although the present invention has been detailed in accordance with the above embodiments, one with ordinary skill in the art should understand that: one can still make modifications to the technical solutions recorded in the above embodiments, or make equivalent substitutions to part or all of the technical features therein; and neither these modifications nor substitutions shall make essence of the corresponding technical solutions depart from the scope of the technical solutions in the embodiments o the present invention.

What is claimed is:

1. A method for preparing pulp for paper with grass straws as raw material, comprising the following steps:
1) adding water or recycled process water into a raw material for pulping to form a mixture, performing a first enzymolysis to the mixture with ligninolytic enzyme to obtain a first enzymolysis product;
2) cooking the first enzymolysis product at a temperature of 80-130° C. to obtain a cooked product;
3) performing a second enzymolysis to the cooked product with hemicellulase to obtain a second enzymolysis product.

2. The method in accordance with claim 1, further comprising step 4): performing dewatering to the second enzymolysis product to obtain a dewatered product, and performing a third enzymolysis to the dewatered product with hemicellulase.

3. The method in accordance with claim 1, wherein, in step 1), a mass ratio of the raw material for pulping to the water or recycled process water is 1:(5-10); enzyme activity of the ligninolytic enzyme is 8,000-12,000 U/g, the amount of the ligninolytic enzyme is 0.05-0.2 kg per 100 kg of the mixture of the raw material for pulping and the water or recycled process water; and temperature of the first enzymolysis is 40-80° C., pH value is 5-8, and enzymolysis time is 0.5-4 h.

4. The method in accordance with claim 2, wherein, in step 1), a mass ratio of the raw material for pulping to the water or recycled process water is 1:(5-10); enzyme activity of the ligninolytic enzyme is 8,000-12,000 U/g, the amount of the ligninolytic enzyme is 0.05-0.2 kg per 100 kg of the mixture of the raw material for pulping and the water or recycled process water; and temperature of the first enzymolysis is 40-80° C., pH value is 5-8, and enzymolysis time is 0.5-4 h.

5. The method in accordance with claim 1, wherein, in step 2), the cooking is conducted at a pH value of 8-12 with a cooking time of 0.5-1.5 h.

6. The method in accordance with claim 2, wherein, in step 2), the cooking is conducted at a pH value of 8-12 with a cooking time of 0.5-1.5 h.

7. The method in accordance with claim 1, wherein, in step 3), the hemicellulase comprises xylanase and mannase, and a mass ratio of the xylanase to the mannase in the hemicellulase is (6-8): (2-4).

8. The method in accordance with claim 2, wherein, in step 3), the hemicellulase comprises xylanase and mannase, and a mass ratio of the xylanase to the mannase in the hemicellulase is (6-8): (2-4).

9. The method in accordance with claim 7, wherein, in step 3), enzyme activities of the xylanase and the mannase are both 8,000-10,000 U/g, the amounts of the xylanase and the mannose are both 0.05-0.2 kg per 100 kg of the cooked product; and the temperature of the second enzymolysis is controlled at 40-70° C., pH value is 8-12, and enzymolysis time is 0.5-4 h.

10. The method in accordance with claim 2, wherein, in step 4), the hemicellulase comprises xylanase and mannase, and a mass ratio of the xylanase to the mannase in the hemicellulase is (4-6):(6-4).

11. The method in accordance with claim 10, wherein, in step 4), enzyme activities of the xylanase and the mannase are both 8,000-10,000 U/g, the amounts of the xylanase and the mannose are both 0.05-0.2 kg per 100 kg of the dewatered product; and the temperature of the third enzymolysis is controlled at 40-70° C., pH value is 8-12, and enzymolysis time is 1-4 h.

12. The method in accordance with claim 10, wherein, in step 4), the third enzymolysis to the dewatered product is conducted with a mixture of hemicellulase and ligninolytic enzyme, wherein enzyme activity of the ligninolytic enzyme is 8,000-12,000 U/g, and the amount of the ligninolytic enzyme is 0.01-0.1 kg per 100 kg of the dewatered product.

13. The method in accordance with claim 11, wherein, in step 4), the third enzymolysis to the dewatered product is conducted with a mixture of hemicellulase and ligninolytic enzyme, wherein enzyme activity of the ligninolytic enzyme is 8,000-12,000 U/g, and the amount of the ligninolytic enzyme is 0.01-0.1 kg per 100 kg of the dewatered product.

14. A system used for the method according to claim 1, comprising a first enzymolysis device, a cooking device and a second enzymolysis device which are sequentially connected; wherein the first enzymolysis device is used for performing the first enzymolysis, the cooking device is used for performing the cooking, and the second enzymolysis device is used for performing the second enzymolysis.
